(19) United States
(12) Patent Application Publication
(10) Pub. No.: US 2024/0379199 A1
(43) Pub. Date: Nov. 14, 2024

(54) SYSTEM AND METHOD FOR PROCESSING MEDICAL DATA

(71) Applicant: iSBERGDATA Corp., Winnipeg (CA)

(72) Inventors: Sadeesh Srinathan, Winnipeg (CA); Andrew Morican, Winnipeg (CA); Harbinder Singh, Toronto (CA)

(21) Appl. No.: 18/316,600

(22) Filed: May 12, 2023

(51) Int. Cl. G16H 10/60

(52) U.S. Cl. CPC G16H 10/60

(57) ABSTRACT

A system and method for processing medical data and more specifically to classifying, deidentifying, and securing medical data is provided. The system and method have a backend processor executing a deidentifying process and a classifying process; a frontend processor executing a data input process; at least one user device providing a personal identifier and the medical data to the data input process; the deidentifying process encrypting the personal identifier to produce an encrypted identifier and generating an independent subject identifier; the classifying process classifying the medical data into a hierarchical classification to produce a hierarchical classified data; and storing the encrypted identifier, the independent subject identifier, and the hierarchical classified data in a database.

### FIELD

This invention is in the field of processing medical data, and more specifically to classifying, deidentifying, and securing medical data.

### BACKGROUND

U.S. Pat. No. 8,731,966 patented by Humedica, Inc. on May 20, 2014 discloses a clinical analytics platform that automates the capture, extraction, and reporting of data required for certain quality measures, provides real-time clinical surveillance, clinical dashboards, tracking lists, and alerts for specific, high-priority conditions, and offers dynamic, ad-hoc quality reporting capabilities. The clinical informatics platform may include a data extraction facility that gathers clinical data from numerous sources, a data mapping facility that identifies and maps key data elements and links data over time, a data normalization facility to normalize the clinical data and, optionally, de-identify the data, a flexible data warehouse for storing raw clinical data or longitudinal patient data, a clinical analytics facility for data mining, analytic model building, patient risk identification, benchmarking, performing quality assurance, and patient tracking, and a graphical user interface for presenting clinical analytics in an actionable format.

U.S. Pat. No. 6,611,846 patented by Medtamic Holdings on Aug. 26, 2003 discloses a method of storage and retrieval of medical information by use of a database that facilitates accurate clinical audit, research and/or presentation activities. Comprehensive patient information may be retrieved based on patient descriptive categories including the anatomy, pathology or clinical presentation, treatment and outcome factors of each case. The categories include data options that may be organized in the form of a hierarchical tree that has branching levels of data options with decreasing specificity. Data from the various levels may be compared, as well as data between individual categories. In some embodiments, selected multimedia data may be accessed based on criteria from data options of the patient descriptive categories.

U.S. Pat. No. 7,490,048 patented by Decapolis Systems LLC on Feb. 10, 2009 discloses a computer-implemented method, including processing, with a processor, a request by a person or entity to access, obtain, change, alter, or modify, an individual's or patient's healthcare record or file which contains healthcare information or healthcare-related information personal to the individual or patient, generating a message containing information regarding the person or entity and/or identification information regarding the person or entity, and containing an actual change, alteration, or modification, sought to be made or made to the individual's or patient's healthcare record or file, and transmitting the message to a communication device of the individual or patient during, concurrently with, at a same time as, or prior to a completion of, an accessing, obtaining, changing, altering, or modifying, of the individual's or patient's healthcare record or file or a processing of the request.

U.S. Pat. No. 10,872,684 patented by John Hopkins University on Dec. 22, 2020 discloses a computer-implemented method may be provided for analyzing and disseminating medical information. The method may include steps performed by one or more processors including, receiving a plurality of patient medical data; aggregating the plurality of patient medical data, wherein access to patient private health information is restricted; receiving a query for medical information; analyzing the aggregated medical data based on the query; producing a result of the query based on the analyzing of the aggregated medical data; and transmitting the result of the query.

These processes, however may be time-consuming and may lack efficiency as data may not be recorded and classified instantaneously.

### SUMMARY

The present invention may allow for, and is not limited to, any and all aspects of combinations, variations, modifications provided herein.

According to an aspect, there is provided a system for processing medical data having: a backend processor executing a deidentifying process and a classifying process; a frontend processor executing a data input process; and at least one user device providing a personal identifier and the medical data to the data input process. The deidentifying process may encrypt the personal identifier to produce an encrypted identifier and may generating an independent subject identifier. The classifying process may classify the medical data into a hierarchical classification to produce a hierarchical classified data. The encrypted identifier, the independent subject identifier, and the hierarchical classified data may be stored in a database. The frontend processor may provide a dashboard to the at least one user device.

The classifying process may further classify the medical data into at least one of: patient data, intervention data, and outcome data. The classifying process may further classify the medical data into at least one of: first contact data, baseline data, admission data, procedure data, and follow-up data.

An encryption key and a decryption key may be stored in a key vault with the independent subject identifier.

The backend processor may execute a reassociation process. The reassociation process may retrieve the decryption key associated with the independent subject identifier; and may decrypt the encrypted identifier. The reassociation process may decrypt demographic data associated with the independent subject identifier.

The hierarchical classification may comprise at least one of: an operation type, a site side location; an incision site; an organ, an action, a lobe, and a segment. The data input process may provide the hierarchical classification.

An analytics process may process the hierarchical classified data for a plurality of patients.

According to another aspect, there is provided a method for processing medical data comprises: receiving a personal identifier and the medical data from at least one user device; encrypting the personal identifier to produce an encrypted identifier; generating an independent subject identifier; classifying the medical data into a hierarchical classification to produce a hierarchical classified data; storing the encrypted identifier, the independent subject identifier, and the hierarchical classified data in a database. The method may provide a dashboard to the at least one user device. The classifying step may classify the medical data into at least one of: patient data, intervention data, and outcome data. The classifying step may classify the medical data into at least one of: first contact data, baseline data, admission data, procedure data, and follow-up data.

The method may store an encryption key and a decryption key in a key vault associated with the independent subject identifier. The method may retrieve the decryption key associated with the independent subject identifier; and decrypting the encrypted identifier. The method may decrypt demographic data associated with the independent subject identifier.

The hierarchical classification may comprise at least one of: an operation type, a site side location; an incision site; an organ, an action, a lobe, and a segment. The data input process May structure the hierarchical classification. The method may process the hierarchical classified data for a plurality of patients.

### DESCRIPTION OF THE DRAWINGS

While the invention is claimed in the concluding portions hereof, example embodiments are provided in the accompanying detailed description which may be best understood in conjunction with the accompanying diagrams where like parts in each of the several diagrams are labeled with like numbers, and where:

FIG. 1 is a high level diagram of a system for processing medical data;

FIG. 2 is a block diagram of an architecture of the system;

FIG. 3 is a high-level flow diagram of the system;

FIG. 4 is a diagram of the users and the data input;

FIG. 5 is a diagram of the data gathering block;

FIG. 6A is a timeline flow of the data gathered; FIG. 6B is an example of a patient flow through a medical system;

FIG. 7 is a detailed diagram of a de-identifying block;

FIG. 8A illustrates a conceptual diagram demonstrating deidentification of personal health data; FIG. 8B illustrated a conceptual diagram demonstrating reassociation of personal health data;

FIG. 9 is an example of classifying data;

FIG. 10A is a flowchart of an authentication component; FIG. 10B is a flowchart of an authorization component; FIG. 10C is a flowchart of an encryption/decryption component;

FIG. 11 illustrates modules and sub-modules;

FIG. 12 illustrates an administration dashboard;

FIG. 13 illustrates an analysis dashboard;

FIG. 14 illustrates a data entry interface;

FIG. 15 illustrates a report dashboard for procedures; and

FIG. 16 illustrates a complications dashboard.

### DETAILED DESCRIPTION

Illustrative embodiments of the invention are described below. The following explanation provides specific details for a thorough understanding of and enabling description for these embodiments. One skilled in the art will understand that the invention may be practiced without such details. In other instances, well-known structures and functions have not been shown or described in detail to avoid unnecessarily obscuring the description of the embodiments. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list. When the word “each” is used to refer to an element that was previously introduced as being at least one in number, the word “each” does not necessarily imply a plurality of the elements, but can also mean a singular element.

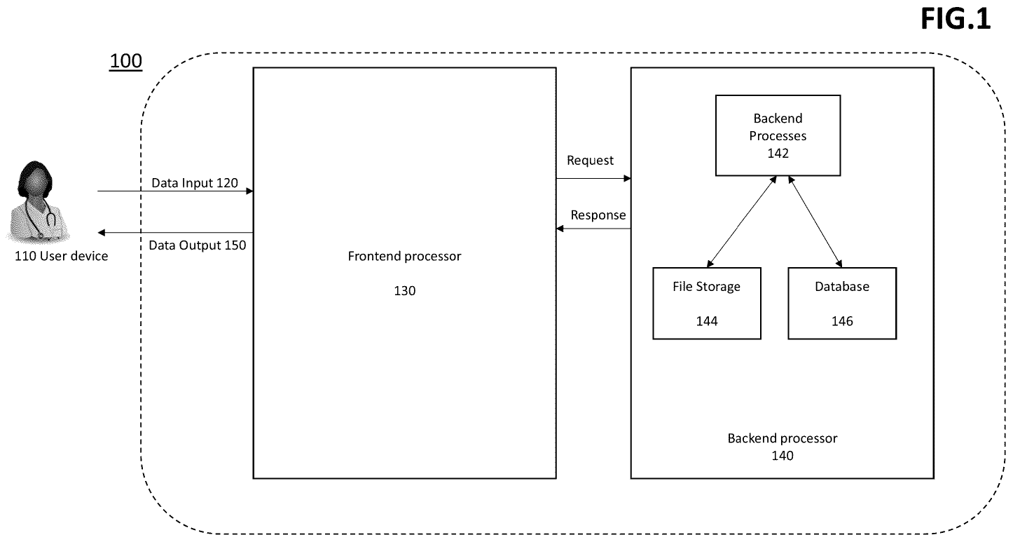

FIG. 1 illustrates a system 100 for processing medical data. The system 100 may include one or more user devices 110 that provide data input process 120 to a front-end processor 130 and may receive data output 150 from the front-end processor 130. The front-end processor 130 may provide the data input 120 to a back-end processor 140 for processing. The back-end processor 140 may execute a backend process module 142 and may store the processed data input in a file storage 144. For example, the user device 110 may provide an X-ray image file to the frontend processor 130, which may in turn be passed onto the back-end processor 140 that stores the X-ray image file in file storage 144. The back-end processor 140 may further include one or more databases 146 to retrieve and/or store information and/or file stored within the file storage 144. Although the aspect shown depicts the file storage 144 and the database 146 as part of the backend processor 140, other aspects may have the file storage 144 and/or the database 146 as processors distinct from the backend processor 140.

The back-end processor 140 may receive one or more requests for data from a user interface (not shown) executing on the front-end processor 130. The requests for data may be transferred over a network (not shown). The back-end processor 140 may then retrieve the data from the database 146 and/or file storage 144 and provide the data to the front-end processor 130 for display and/or processing by the front-end processor 130. The data and/or the processed data may be displayed via a data output 150 on the user interface.

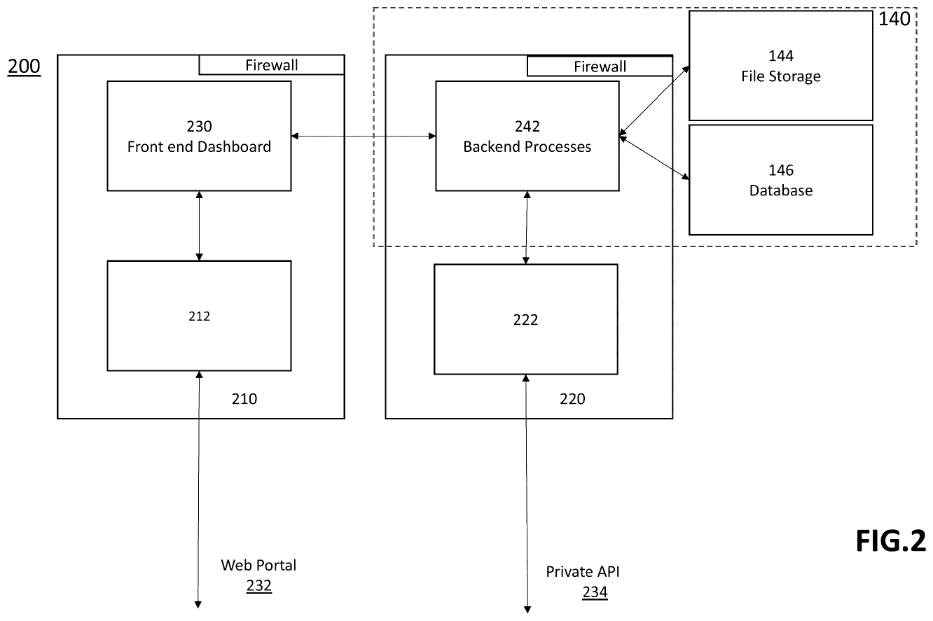

As shown in FIG. 2, a software architecture 200 of the system 100 may include a public platform 210 and a private platform 220. In this aspect, the public platform 210 may execute on the front-end processor 130 and the private platform 220 may execute on the back-end processor 140. The public platform 210 may communicate over the Internet using a web portal 232 or the like. The private platform 220 may provide a private Application Programming Interface (API) 234 to communicate locally over an Intranet (and/or over the Internet via a Virtual Private Network (VPN)) with a private server 222. The public platform 210 and/or the private platform 220 may provide one or more firewalls.

The public platform 210 may execute a front-end dashboard 230 that may receive one or more user inputs via a public server 212. The public server 212 may provide the web portal 232 that enables access over the Internet. The web portal 232 may receive instructions and data from the remote computer 110. One such instruction may be to generate one or more output reports. The output reports may comprise medical reports and/or analytical reports. The public server 212 receives the instruction to generate a report. The public server 212 may interface with the front-end dashboard 230 in order to retrieve data from the private platform 220 via a backend process API 242. Depending on the request, the backend process API 242 may determine whether the local database 146 and/or the file storage 144 is being accessed. The backend process API 242 may then process the accessed data and/or files prior to providing the requested data to the front end dashboard 230.

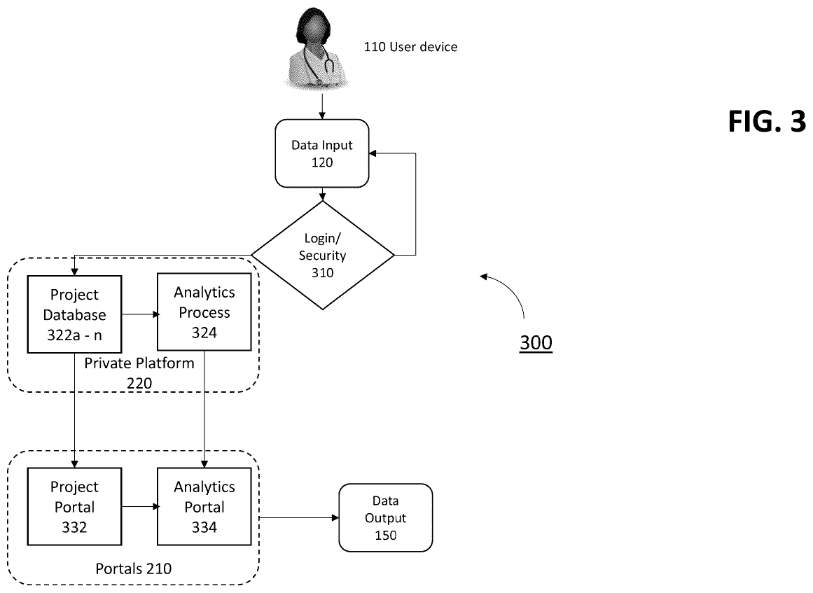

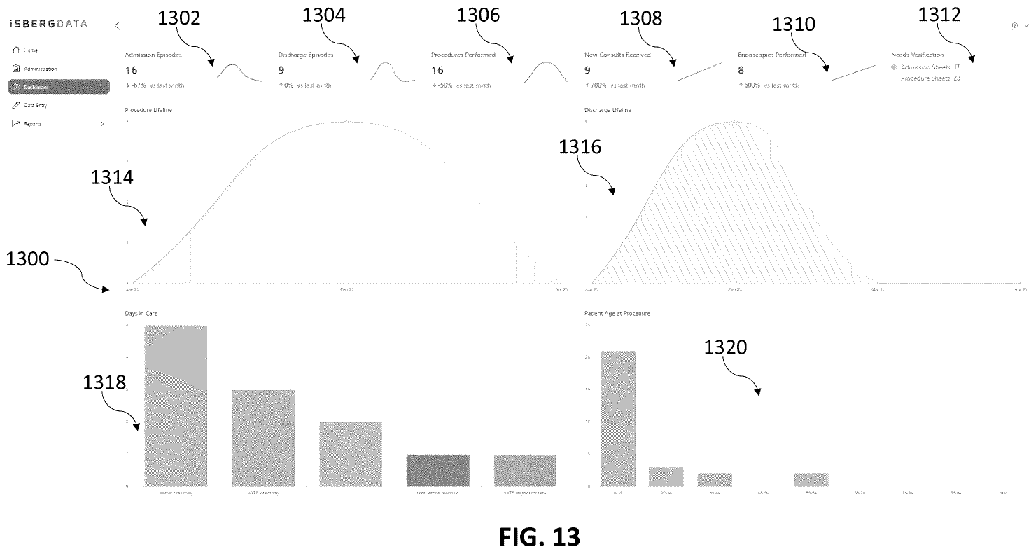

With respect to FIG. 3, the users devices 110, such as used by a medical professional, may provide data into a data input process 120. A security process 310 may receive data from the data input process 120. When one or more security conditions of the security process 310 are not met, the security process 310 returns to the data input process 120. For example, when the username and/or password do not match or if access is by an unauthorized device. When the security conditions are met, the security process 310 may provide access to one or more project databases 322a-n executing on the private platform 220. In this manner, the users devices 110 may provide new medical data or edit medical data of one or more patients. An analytics process 324 may perform analysis on the project databases 322a-n to generate analysis data. For example, the analytics process 324 may provide one or more statistics, such as admission episodes, discharge episodes, procedures performed, new consultations received, procedure lifeline, discharge lifeline, days in care, patient age at procedure, procedure summaries and the like. An example of this analytics output is provided in FIGS. 13, 15, and 16.

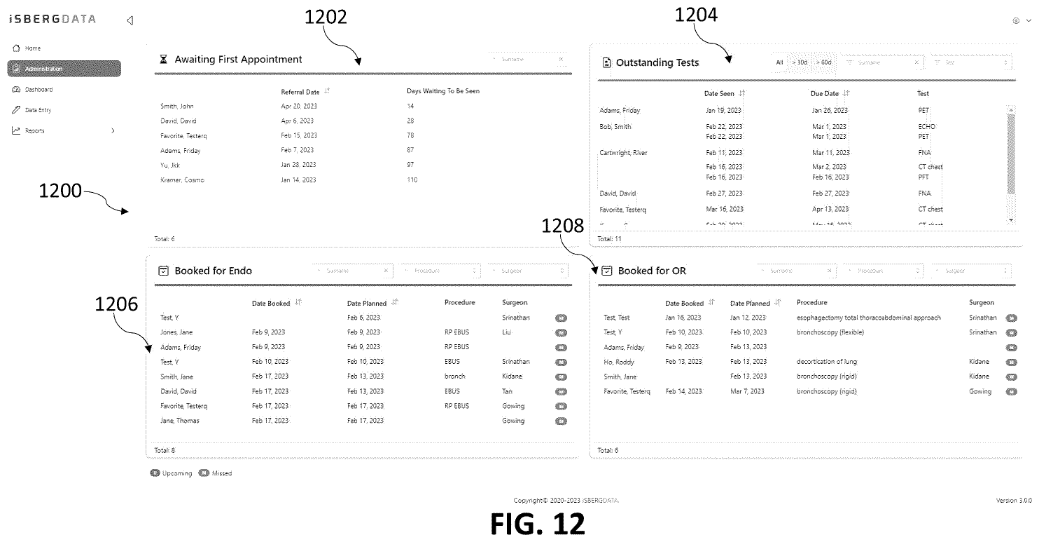

The private platform 220 may be access from one or more portals executing on the public platform 210. In this aspect, a data output process 150 may receive analysis data via an analytics portal 334. The analytics portal 334 may retrieve project data from the project databases 322a-n via a project portal 332. In this aspect, the analytics process 324 may process only the data from the selected project database 322a-n from the project portal 332. The data provided by the analytics portal 334 may then be output using the data output process 150 in the form of the reports, analysis tables, or the like. Examples of the data output may be seen in FIGS. 12 to 16 described in further detail below.

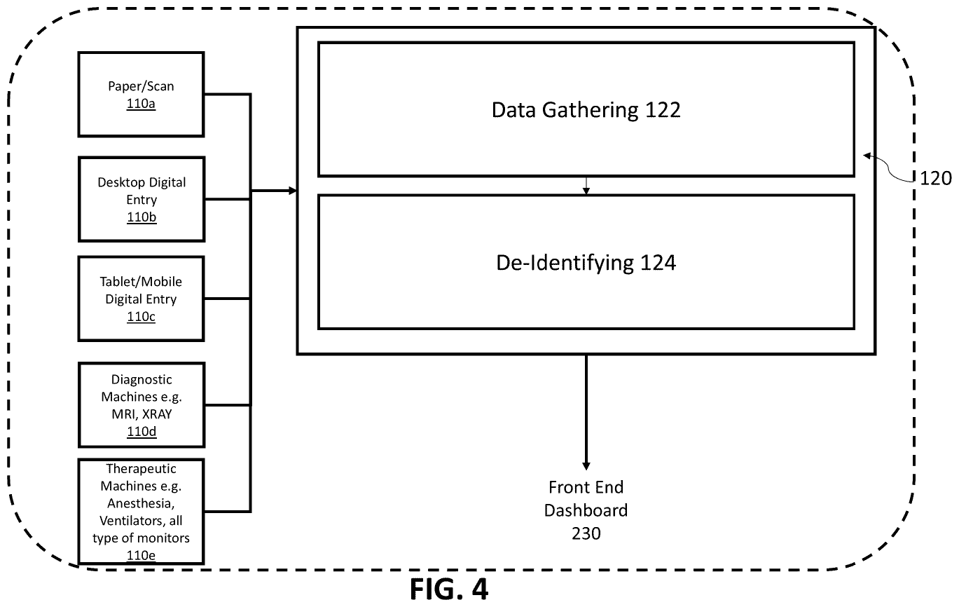

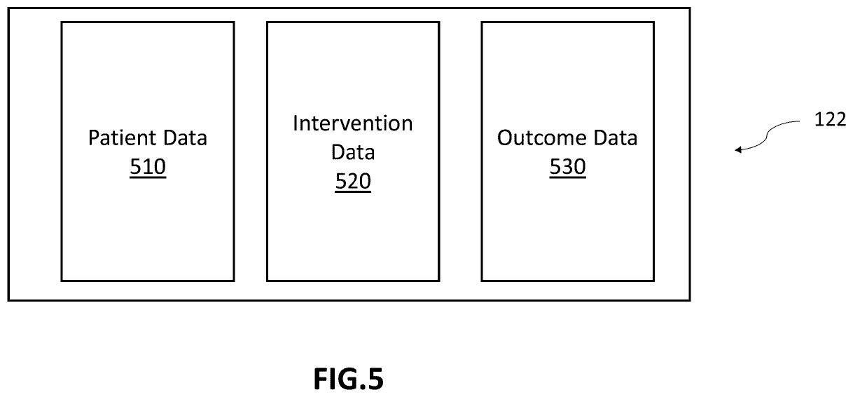

With reference particularly to FIGS. 4 and 5, the data input process 120 may include data retrieved in real-time via scanning one or more paper records 110a, digital entry 110b, etc. using desktops, laptops, or the like, personal devices 110c such as mobiles, tablets or the like, diagnostic machines 110d such as MRIs, X-rays, etc. and/or therapeutic machines 110e such as anesthesia, ventilators, or any type of monitor(s). Data may be gathered with a data gathering process 122 and de-identified with a de-identification process 124.

The data gathered may include patient data 510, intervention data 520, and/or outcome data 530. The de-identification process 124 may focus primarily on removing patient data associated with identity of the patient while maintaining the intervention data 520 and the outcome data 530. The patient data 510 may include, but is not limited to, gender, socioeconomic status, symptoms, comorbidities, medication use, diagnosis, urgency, anatomic details, referral information, and/or anatomy. The intervention data 520 may include, but is not limited to, date, anatomy, implants, trainees, times, findings, pump run, surgery specific information, and/or success of procedure. The outcome data 530 may include, but is not limited to, symptoms, anatomy, Quality of Life, events, social impact, work, analgesic use, other prescriptions received, and/or imaging/lab data.

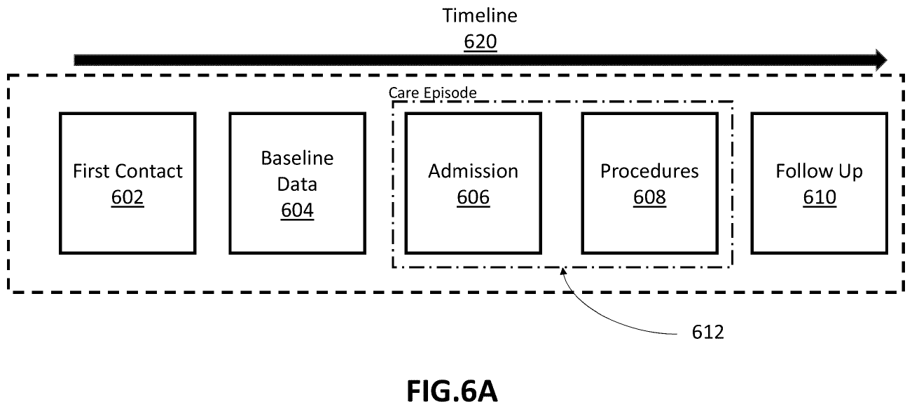

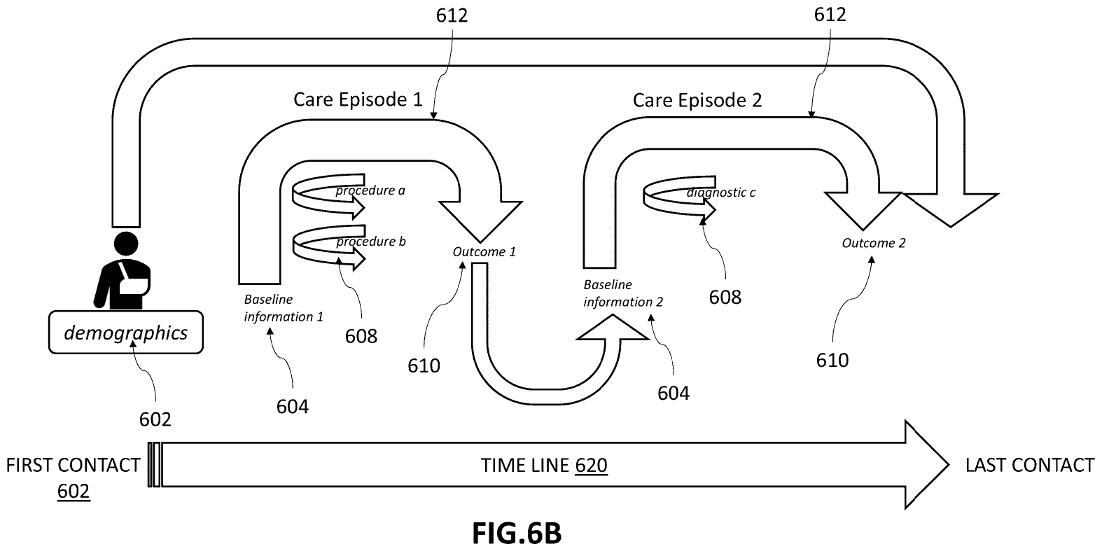

As shown in FIGS. 6A and 6B, the data gathering process 122 may involve many interactions with the patient-health system 100 over a period of time known as a patient timeline 620. In this aspect, the gathered data may be recorded in real-time by a physician and/or another user with authorized access provided by the security process 310. The gathered data may be classified as first contact data 602, baseline data 604, admission episode data 606, procedure data 608, and/or follow up data 610, which may correspond to the outcome data 530. The first contact data 602 may involve patient data 510. Generally, the admission episode data 606 and the procedure data 608 may be collectively referred to as a care episode data 612 or intervention data 520. At each of these interactions 602, the gathered data may be processed by the de-identification process 124 in order to remove any identifiable data and ensure uniqueness of the patient in order to properly associate new patient data 510, new intervention data 520, and/or new outcome data 530 with the unique patient.

A particular example is shown in FIG. 6B. A client may have first contact data 602 entered into the data input process 120. As depicted, the client has undergone two care episodes 612 wherein the first care episode involved two procedures. The first care episode generates baseline data 604, two procedure data 608, and one outcome data 610. The outcome data 610 may serve as a basis for the baseline data 604 of the second care episode. The second care episode involves one diagnostic procedure generating diagnostic data and one outcome data 610.

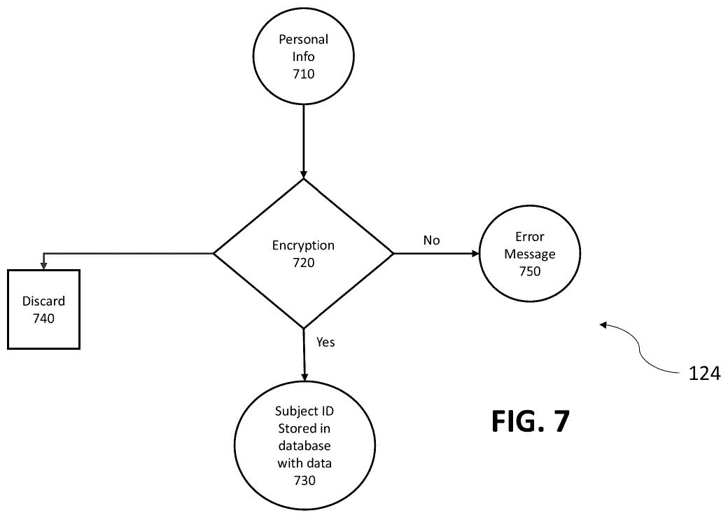

Turning to FIG. 7, the de-identifying process 124 may involve an identifying process 710 that determines personal identifying data. The personal identifying data may then be encrypted by an encryption process 720 to produce a unique identifier. The unique identifier may be stored in the project databases 322a-n along with data that is not personally identifying. The personal identifying data may then be discarded using a discard process 740. If the encryption fails, an error message is output at 750. Alternatively, the personal identifying information and the unique identifier may be stored on the local database 146 along with the data that is not personally identifying.

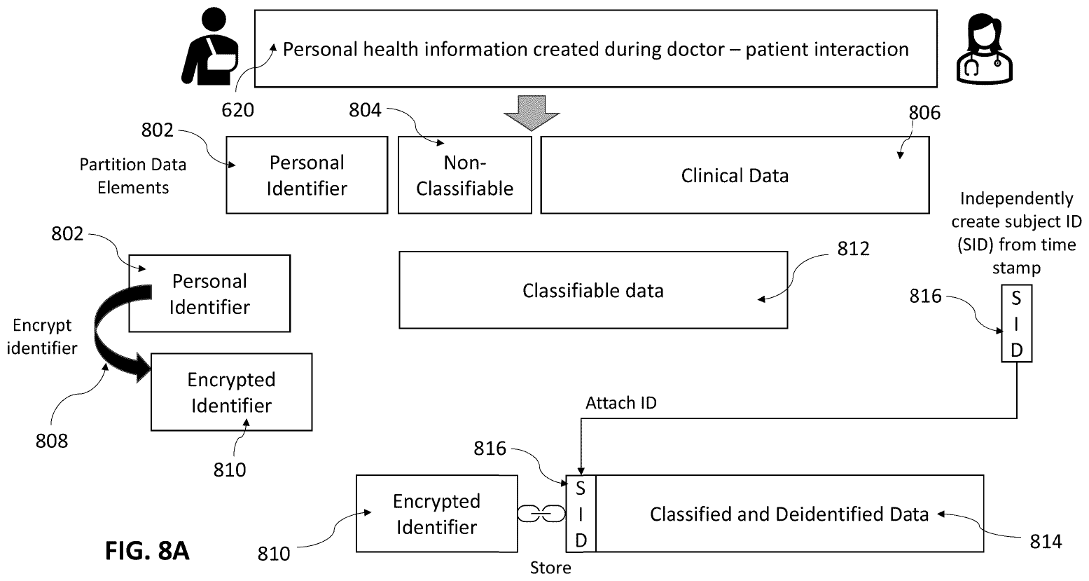

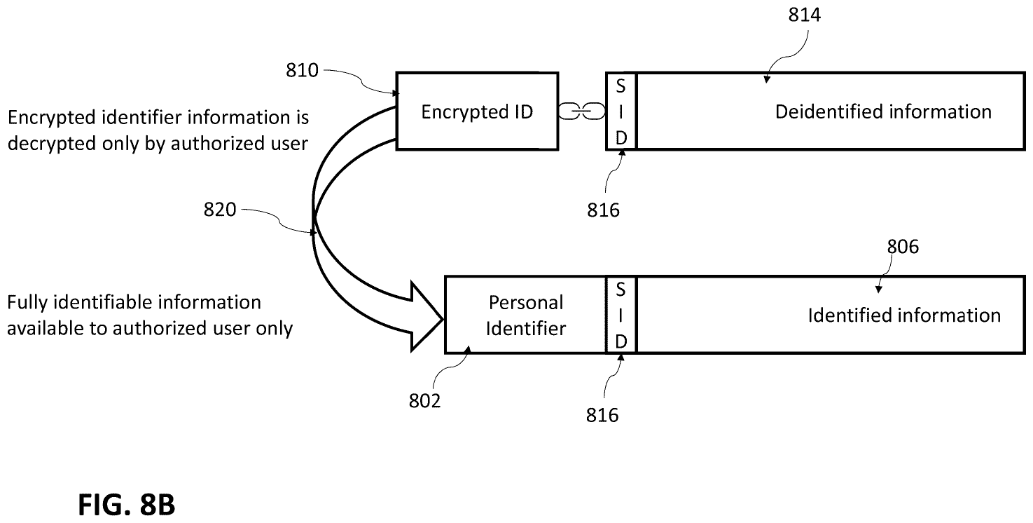

FIG. 8A further details the data input and recorded during interactions between the user device 110 and a client during the patient timeline 620. During the patient timeline 620, users 110 may enter personal identifier 802, such as for example a health insurance number or may scan biometric data. The personal identifier 802 may be converted using an encryption module 808 into an encrypted identifier 810. When the personal identifier 802 is encrypted, an independently created subject identifier (SID) 816 may be generated using a time stamp. The SID 816 may be involved in generating the encrypted identifier (e.g. by acting as part of a hash). A portion of the provided data from the patient timeline 620 may comprise non-classifiable data 804. The non-classifiable data 804 may be stored on the local database 146 as the data 804 may not provide useful information to researchers investigating the project database 322a-n. In other aspects, the non-classifiable data 804 may be discarded. Another portion of the provided data from the patient timeline 620 may be classifiable data 806, which may be entered using a classification module 812 in order to produce deidentified and classified data 814. The classifiable data 806 may comprise patient information 510, intervention information 520, and outcome information 530. The encrypted identifier 810 may then be combined with the SID 816 and the deidentified and classified data 814 to be stored in the project database 322a-n. In this manner, the identity of the data cannot be traced back to the patient's identity.

In an example, the personal identifier 802 may comprise biometric data from a biometric scan, such as an iris scan, fingerprint scan, or the like. The biometric data may then be encrypted using the encryption process 720, such as a one way hash. The biometric data may then be securely discarded 740. In this aspect, no other identifiable information is necessary to be provided. Through this de-identifying process 124, risks associated with identity and access to healthcare may be reduced and/or eliminated. For example, rather than providing a name to the healthcare provider, the biometric scan generates the unique identifier that is not associated with the identity of the patient. As the medical history without identifying data is stored on the project databases 322a-n, the patient may be treated without risk of their identity being discovered. The medical practitioner may view the medical history data and treat the patient without knowledge of the identity of the patient. If the medical practitioner attempts to provide identifying data, the de-identifying process 124 may remove the identifying data in order to protect the patient.

After determining the SID 816 and the encrypted identifier, the classified data may be recorded in real-time into using a hierarchical taxonomy of up-down concepts. The classified data may be tagged with metadata such that at the time of recording, text may be auto populated based on priority in the hierarchy and predetermined information from the various databases accessed.

According to some aspects, as only the SID 816 and classified data 814 may need to be provided, an amount of data to be transferred to the project databases 322a-n may be considerably less than that for electronic medical records (EMRs) that require identifiable data to be provided. In some instances, prior EMRs require transfer of the entire medical record. In areas with limited Internet connectivity, these prior EMRs may be slow as the amount of data to be transferred exceeds the capabilities of the Internet connection and/or expensive as remote communities typically pay for data usage. As described herein, the entire medical record and identifiable data do not have to be transferred. As the classified data 814 has also been deidentified, encryption of the data 814 does not have to be performed, which may further increase the speed of storing and/or retrieving the classified data 814. According to some aspects, the classified data may also be compressed.

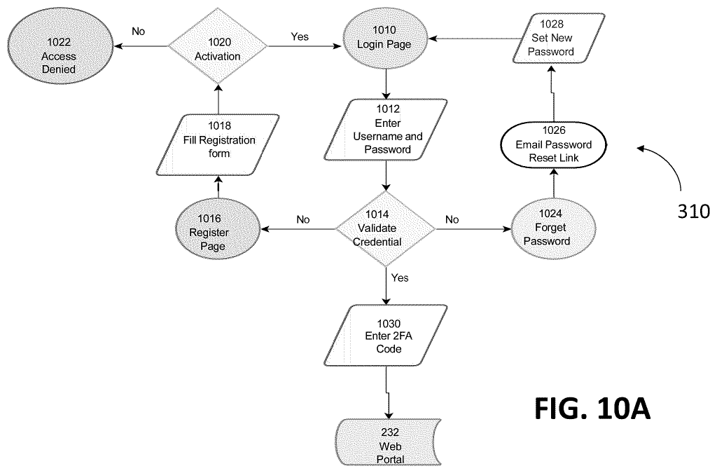

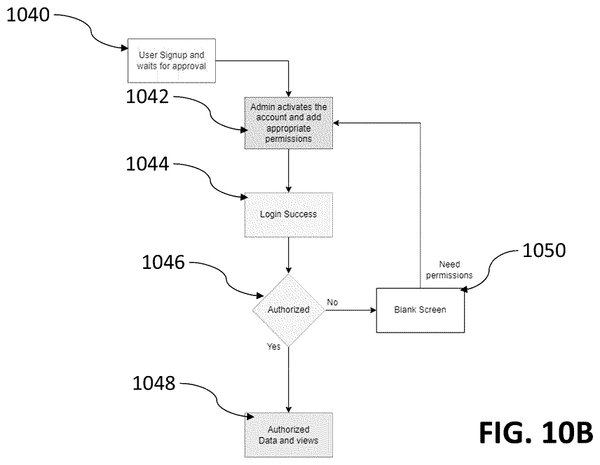

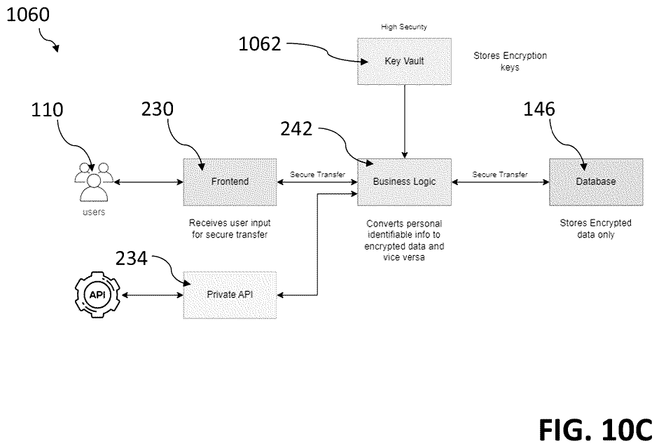

Turning to FIG. 8B, when the deidentified and classified data 814 is to be reassociated with the patient, a reassociation process involves an authorized user may be provided with a decryption key in order to decrypt 820 the encrypted identifier 810 in order to retrieve the personal identifier 802. The decryption process 820 may be performed when the user interface is required to display demographic information of the patient (e.g. name, date of birth, etc.). This process may be further described below with reference to FIG. 10C.

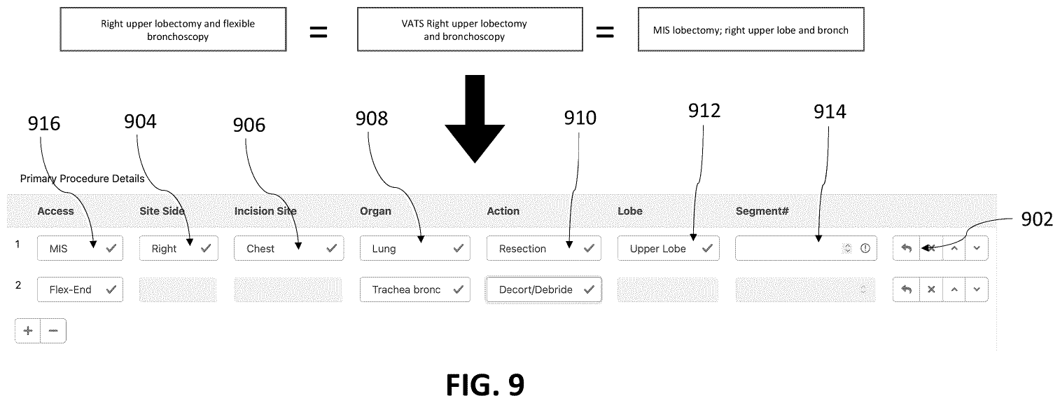

As shown in FIG. 9, a categorized input user interface 902 for an operation may be defined by how the operation is performed. The user interface 902 may present a number of dropdowns for classifying the operation. For example, the operation dropdown 916 may present options of minimally invasive, open procedure, minimally invasive converted to open, and the like. Once the operation type dropdown 916 has been selected, a site side location dropdown 904 may be created and/or auto-populated. The site side location dropdown 904 may determine an incision site dropdown 906. Based on the incision site dropdown 906, an organ dropdown 908 may be populated with one or more organs located near the incision site. For example, when the incision site dropdown 906 is selected for “chest”, the organ dropdown 908 may be populated with lung and heart. An action dropdown 910 may be presented with types of surgical actions performed on that organ, such as resection, debridement, biopsy, repair, and the like. Where appropriate for the selected organ, a lobe dropdown 912 and/or a segment number dropdown 914 may be presented. After gathering or recording the data into local portal 334, user devices 110 may login to server 320. The process of logging into server 320 is described with reference to FIGS. 10A-C hereinafter.

As shown in FIG. 10A, a user device 110 may access a login page 1010 and provide a user name and a password 1012. The private server 220 may then execute instructions to validate credentials 1014, and when the credentials are validated, the user device 110 may be prompted to enter a two-factor authentication 1030 to permit access to the web portal 232. When the validate credentials process 1014 determines the username and password are new, a registration process 1016 may provide a registration form 1018 and/or execute an activation process 1020 to set up two-factor authentication 1030. When validating credentials 1014 fails, the user device 110 may select a forget password process 1024. An email with password reset link may be sent 1026, and user device 110 may set new password 1028. At which point the user device 110 may re-access login page 1010. While accessing the private server 220, the user interface 902, such as shown in FIG. 9, may be provided. When the interaction is completed, user device 110 may transmit the data to the private server 220.

Turning to FIG. 10B, once the user device 110 has been authenticated, the user device 110 waits for approval at step 1040. An administrator may then activate the account and add one or more permissions at step 1042. Once the permissions have been added, the user device 110 may successfully login at step 1044. An authorization process 1046 may then determine one or more authorized data views in order to provide the authorized data and views 1048 to the user device 110. When the user device 110 is not authorized, a blank screen 1050 may be provided and a notice may be provided to the administrator to add permissions at step 1042.

A reassociation process 1060 is shown in FIG. 10C. An authenticated and authorized user device 110 accesses the front end 230 and requests a secure transfer using the SID 816. The front end 230 initiates the secure transfer with the backend process 242. The backend process 242 retrieves the encrypted data from the database 146 using the SID 816. The backend process 242 may then retrieve a decryption key from a key vault 1062 corresponding to the SID 816. The backend process 242 decrypts the encrypted data and provides the decrypted data to the user device 110.

When identifiable data is to be stored in the database 146 from the user device 110, the front end 230 provides the SID 816 with the identifiable data to the backend process 242. The backend process 242 then retrieves an encryption key from the key vault 1062 corresponding to the SID 816. The backend process 242 may then encrypt the identifiable data. The SID and the encrypted data are then provided to the database 146 for storage.

According to some aspects, the decryption key and the encryption key may be stored on the user device 110 and may be provided to the backend process 242 via the private API 234.

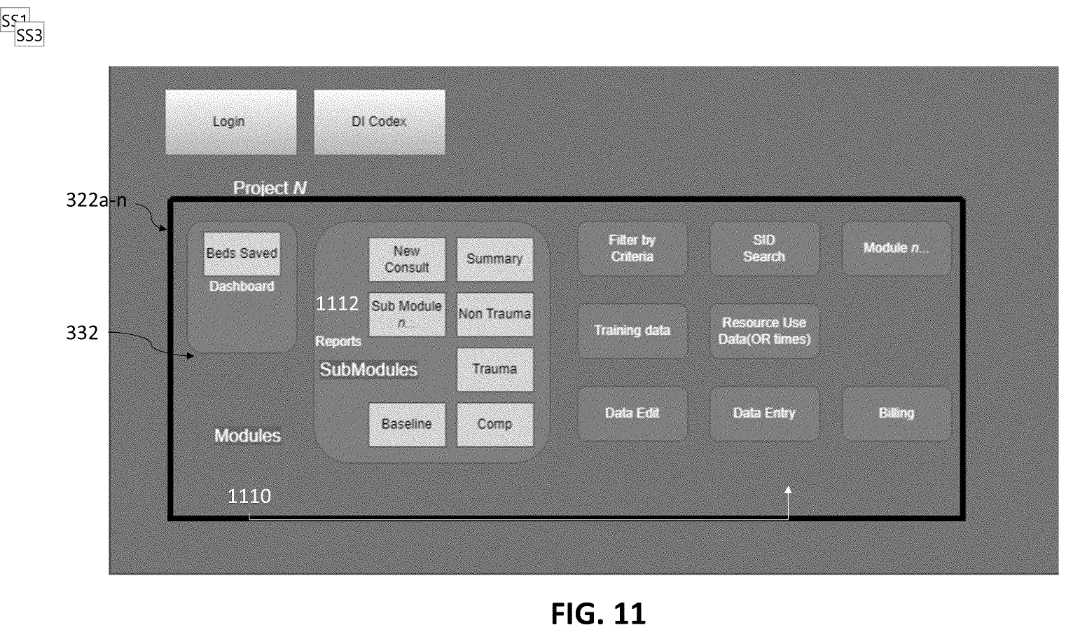

Turning to FIG. 11, each project 322a-n may comprise a project portal 332 (e.g. dashboard) that may present aggregated data, such as a number of beds saved. The project 322a-n may provide a number of report generating sub-modules 1112. The report generating sub-modules 1112 may provide new consult reports, summary reports, non-trauma reports, trauma reports, baseline reports, and comp reports. The project 322a-n may also comprise modules 1110 that allow filtering by criteria, training data, data editing, data entry, resource use, SID 816 search, and billing.

As shown in FIG. 12, an administration dashboard 1200 is shown. The administration dashboard 1200 may provide a list of patients awaiting their first appointment 1202, a list of outstanding tests 1204, a list of patients booked for endoscopic procedure 1206, and/or a list of patients booked for an operating room 1208. Each of the lists may provide relevant data associated with each list. For example, the list of patients awaiting their first appointment 1202 may list the patient name, referral date, and days waiting to be seen. The list of outstanding tests 1204 may provide the patient name, date seen, due date for the test, and a type of the test. The data presented in the administration dashboard comprises identifiable data that has been decrypted by the backend process 242.

As shown in FIG. 13, an analysis dashboard 1300 may provide a plurality of comparisons of a current month to a previous month. These comparisons may comprise admission episodes 1302, discharge episodes 1304, procedures performed 1306, new consults received 1308, and endoscopies performed 1310. Below these comparisons are four plots of the procedure lifeline 1314, discharge lifeline 1316, days in care 1318, and patient age at procedure 1320. The data presented in the analysis dashboard 1300 may be anonymized, aggregate data.

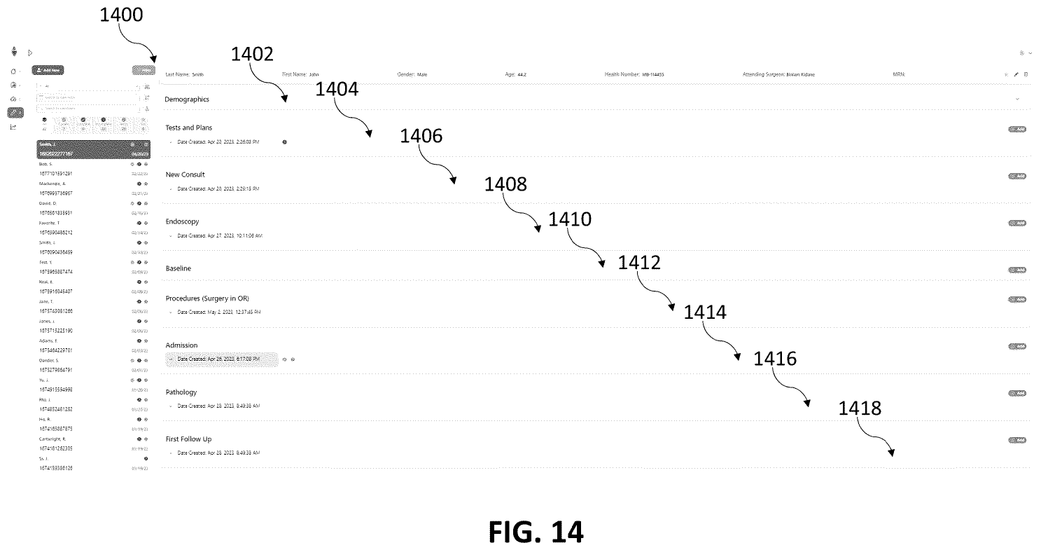

Turning to FIG. 14, a data entry interface 1400 is provided for entering patient data. For each patient, a number of data entry sections may be provided. In this example, the data entry sections comprise: demographic 1402, tests and plans 1404, new consult 1406, endoscopy 1408, baseline 1410, procedures (surgery in OR) 1412, admission 1414, pathology 1416, and first follow up 1418. By categorizing the patient data in this manner, the data de-identifying process 124 may easily remove the demographics data 1402 before providing the remaining data entry sections to the project databases 322a-n. The data de-identifying process 124 may also process text fields of the remaining sections 1404, 1406, 1408, 1410, 1412, 1414, 1416, and 1418 for any identifying data such as any data found in the demographics data 1402, such as name, street address, etc.

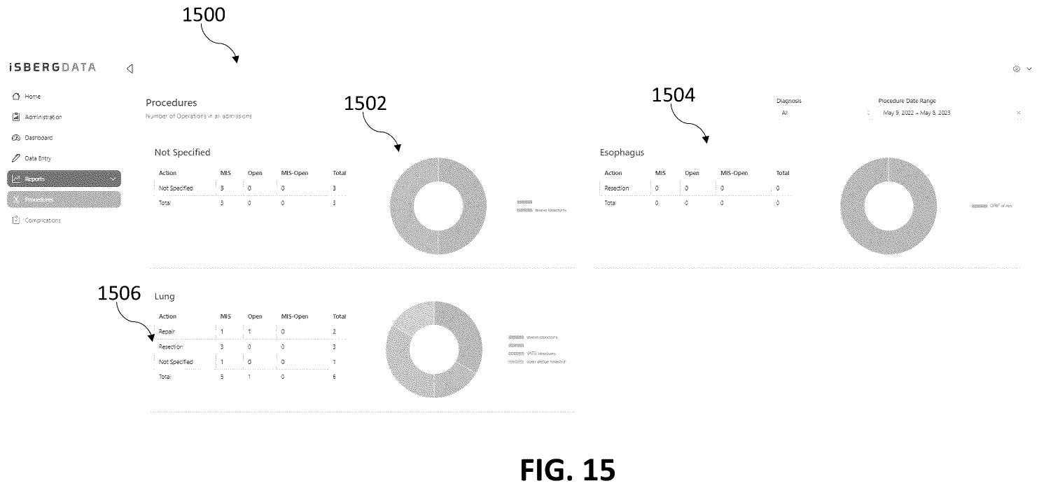

As shown in FIG. 15, a report dashboard for procedures 1500 provides an analysis for each of the procedures present in the project databases 322a-n. In this example, three procedure analyses 1502, 1504, 1506 are presented. Other aspects may have fewer or more procedure analyses. In this aspect, the procedure analysis 1506 may comprise a procedure on the lungs. The procedure analysis 1506 provide a list of the action performed (e.g. repair, resection, none specified) and a type of surgery (e.g. minimally invasive surgery, open, or minimally invasive surgery that becomes open surgery). A graph may be presented for each of these procedures. As the categorized input user interface 902 is used to enter the data, the procedure analysis 1506 is facilitated in an improved manner. The data presented in the report dashboard 1300 may be anonymized, aggregate data.

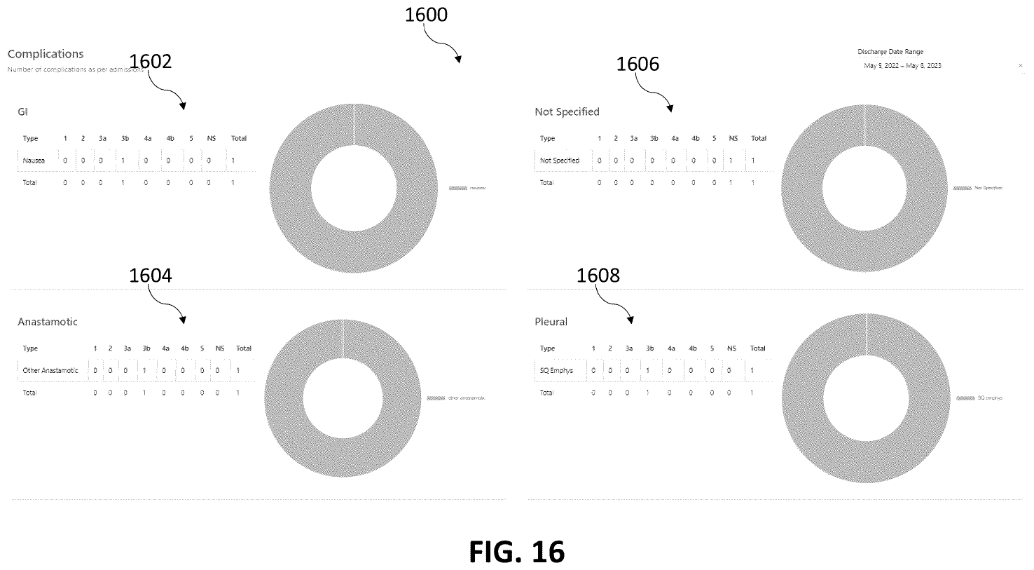

Similarly, FIG. 16 illustrates a complications dashboard 1600 which may likewise be entered using a categorized input user interface 902. In this example shown, four complications 1602, 1604, 1606, 1608 are presented. In this aspect, the four complications are gastroenterology complications 1602, anastomotic complications 1604, not specified complications 1606, and pleural complications 1608. The data presented in the complications dashboard 1600 may be anonymized, aggregate data.

According to an aspect, the anonymized, aggregate data may be publicly available to user devices 110 without authentication and/or authorization being performed.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

The foregoing is considered as illustrative only of the principles of the invention. Further, since numerous changes and modifications will readily occur to those skilled in the art, it is not desired to limit the invention to the exact construction and operation shown and described, and accordingly, all such suitable changes or modifications in structure or operation which may be resorted to are intended to fall within the scope of the claimed invention.

1. A system for processing medical data comprising:
a backend processor executing a deidentifying process and a classifying process;
a frontend processor executing a data input process;
at least one user device providing a personal identifier and the medical data to the data input process;
the deidentifying process encrypting the personal identifier to produce an encrypted identifier and generating an independent subject identifier;
the classifying process classifying the medical data into a hierarchical classification to produce a hierarchical classified data; and
storing the encrypted identifier, the independent subject identifier, and the hierarchical classified data in a database.

2. The system for processing medical data according to claim 1, further comprising: the frontend processor providing a dashboard to the at least one user device.

3. The system for processing medical data according to claim 1, wherein the classifying process further classifies the medical data into at least one of: patient data, intervention data, and outcome data.

4. The system for processing medical data according to claim 1, wherein the classifying process further classifies the medical data into at least one of: first contact data, baseline data, admission data, procedure data, and follow-up data.

5. The system for processing medical data according to claim 1, wherein an encryption key and a decryption key are stored in a key vault with the independent subject identifier.

6. The system for processing medical data according to claim 5, further comprising: the backend processor executing a reassociation process;
the reassociation process comprises: retrieving the decryption key associated with the independent subject identifier; and decrypting the encrypted identifier.

7. The system for processing medical data according to claim 6, wherein the reassociation process further comprises: decrypting demographic data associated with the independent subject identifier.

8. The system for processing medical data according to claim 1, wherein the hierarchical classification comprises at least one of: an operation type, a site side location; an incision site; an organ, an action, a lobe, and a segment.

9. The system for processing medical data according to claim 1, wherein the data input process provides the hierarchical classification.

10. The system for processing medical data according to claim 1, further comprises an analytics process processing the hierarchical classified data for a plurality of patients.

11. A method for processing medical data comprises:
receiving a personal identifier and the medical data from at least one user device;
encrypting the personal identifier to produce an encrypted identifier;
generating an independent subject identifier;
classifying the medical data into a hierarchical classification to produce a hierarchical classified data;
storing the encrypted identifier, the independent subject identifier, and the hierarchical classified data in a database.

12. The method for processing medical data according to claim 11, further comprises:
providing a dashboard to the at least one user device.

13. The method for processing medical data according to claim 11, wherein the classifying classifies the medical data into at least one of: patient data, intervention data, and outcome data.

14. The method for processing medical data according to claim 11, wherein the classifying classifies the medical data into at least one of: first contact data, baseline data, admission data, procedure data, and follow-up data.

15. The method for processing medical data according to claim 11, further comprises: storing an encryption key and a decryption key in a key vault associated with the independent subject identifier.

16. The method for processing medical data according to claim 15, further comprises: retrieving the decryption key associated with the independent subject identifier; and decrypting the encrypted identifier.

17. The method for processing medical data according to claim 16, further comprises: decrypting demographic data associated with the independent subject identifier.

18. The method for processing medical data according to claim 11, wherein the hierarchical classification comprises at least one of: an operation type, a site side location; an incision site; an organ, an action, a lobe, and a segment.

19. The method for processing medical data according to claim 11, wherein the data input process structures the hierarchical classification.

20. The method for processing medical data according to claim 11, further comprises: processing the hierarchical classified data for a plurality of patients.
